(19) United States
(12) Patent Application Publication
(10) Pub. No.: US 2007/0173719 A1
(43) Pub. Date: Jul. 26, 2007

(54) Method and system for representing an examination region of a subject supplemented with information related to the intracorporeal influence of an agent

(76) Inventor: Sultan Haider, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/486,680

(22) Filed: Jul. 14, 2006

(30) Foreign Application Priority Data
Jul. 14, 2005 (DE) 10 2005 032 963.2

(51) Int. Cl. A61B 6/00

(52) U.S. Cl. 600/431

(57) ABSTRACT

In a method and system for representation of at least one examination region of an examination subject, together with at least one item of information relating to the intracorporeal influence of at least one agent to be administered to the examination subject, which information is to be retained for a subsequent evaluation, a whole-body overview image of the examination subject is generated, a data set is acquired (with at least one imaging medical examination apparatus) that is associated with the examination region and the acquired examination region is mapped on the whole-body overview image, a time-related determination of at least one item of information relating to the intracorporeal influence of the agent in the examination region is made and the information is mapped to the examination region in the whole-body overview image; and the whole-body overview image, supplemented in this manner, is represented on an image reproduction device.

### BACKGROUND OF THE INVENTION

1. Field of the Invention

The present invention concerns a method for representing an examination region of an examination subject together with at least one item of information related to the intracorporeal influence of an agent to be retained for a subsequent evaluation.

2. Description of the Prior Art

The production of an image exposure of an examination region of a patient with an imaging medical examination apparatus is often not sufficient for planning a therapy or for a comprehensive diagnosis. Specific agents (such as, for example, contrast agents, biomarkers or medicines) often must additionally be administered, the behavior of which in the body of the patient or in the examination region provides information about existing illnesses or is significant in the framework of a treatment. This is particularly true for molecular imaging, which is under extended development in which biochemical processes in the body of an examination subject are shown.

Such a representation of the intracorporeal influence of an agent administered or to be administered to the examination subject serves, for example, to support gene therapy to be implemented, in which the introduction of the gene into the body and the distribution or expression thereof can be tracked with various imaging medical examination apparatuses (such as magnetic resonance tomography or positron emission tomography). Another example is the administration of stem cells which can be marked with contrast agents detectable by magnetic resonance tomography in order to track their path in the body. Apart from their use to improve an image representation, contrast agents can additionally serve therapeutic purposes, for example in the imaging of structures known as “microspheres” with a hollow inner area that offers space for a further agent. The agent transported within the contrast agent and serving for treatment is released only when the surrounding shell is destroyed by an ultrasonic wave upon the application of high-intensity, focused ultrasound.

The use of these novel types of diagnosis creation or therapy that are in development has previously been associated with high costs, a high time expenditure and a number of uncertainties, since the possibilities for monitoring the respective processes are very limited.

### SUMMARY OF THE INVENTION

An object of the present invention is to provide a method and an apparatus for representing an examination region of an examination subject, to whom an agent has been administered that interacts with the subject, that allow information regarding the influence of the agent on the subject to be included in the representation of the examination region, wherein the aforementioned disadvantages associated with conventional techniques and systems are avoided.

This object is achieved in accordance with the invention by a method of the aforementioned type wherein a whole-body overview image of the examination subject is generated, a data set is acquired (with at least one imaging medical examination apparatus) that is associated with the examination region and the acquired examination region is mapped on the whole-body overview image, a time-related determination of at least one item of information relating to the intracorporeal influence of the agent in the examination region is made and the information is mapped to the examination region in the whole-body overview image, and the whole-body overview image, supplemented in this manner, is presented on an image reproduction device.

In the inventive method, a display tool is thus provided that is based on whole-body imaging. For this purpose, a whole-body overview image is initially created that is sufficiently precise in order to subsequently show (based on this platform) the examination region as well the behavior of an agent, thus generally the situation before the uptake of the agent by the patient, as well as after.

A data set with regard to an examination region is subsequently acquired (the examination region naturally can encompass the entire body of the examination subject), for which an imaging medical examination apparatus is used. The acquired image data are at least partially mapped to the whole-body overview image, which can be a two-dimensional or three-dimensional image.

Furthermore, at the same time, or before or after the acquisition of the data set, information is determined with regard to the behavior of an agent in the examination region, or generally in a larger or smaller sub-region of the entire body with a relation to the examination region. The determination of this information can concern both the situation in the body of the patient before the agent was administered, and the situation after the administration of the agent, if applicable up to a complete decomposition of the agent. The determination of these one or more items of information relating to specific times, or to a time period, can ensue by the implementation of measurements on the examination subject or on the basis of simulations or data of earlier treatments that can be used as an alternative to or an addition to the measurement in order to thus reduce the stress to and possible danger for the patient.

The item or items of information relating to the influence of the agent are likewise mapped to the examination region in the whole-body image. A mapping to another sub-region of the whole body of the patient with a corresponding relation to the examination region is also possible if applicable, such that the aforementioned mapping to the examination region is broadly understood in this sense.

The whole-body overview image supplemented in this manner is ultimately shown on an image reproduction device such as a monitor or a display.

The inventive method provides a tool with which it is possible (in particular given pathological findings in different regions of the body, thus given multiple findings or suspicious facts that suggest an examination of different examination regions) to simultaneously show these examination regions with the effect of an administered agent (such as a medicine). For this purpose, a whole-body overview image is used in which (by repetition of the steps of the method as specified) the acquired image data can present the information about specific findings or form an overview of the present anatomy, and the time-related influences or effects or merely the behavior of agents administered to the patient are shown for selected examination regions.

The representation on the basis of the whole-body platform can be used, for example, for the development of clinically-relevant imaging biomarkers in cancer treatment or for the development as well as monitoring of specific contrast agents, in particular given processes in connection with cancers. The development of agents that can be used in combination for the diagnosis and therapeutic purposes can be advanced by the specified method for representation on the basis of a whole-body overview image that allows a comprehensive orientation.

The whole-body overview image can be created by the same imaging medical examination apparatus used for acquisition of the data set. For example, given the acquisition of a data set with a computed tomography apparatus, an overview scan can initially be created, and the whole-body overview image can be derived from the overview scan data.

Furthermore, the whole-body overview image can be generated by an imaging medical examination apparatus and/or at least one camera and/or an anatomical atlas and/or a computer program. A magnetic resonance apparatus, a computed tomography apparatus, a conventional x-ray apparatus or even an ultrasound apparatus, a positron emission tomography (PET) apparatus and the like are suitable as imaging medical examination apparatuses for generation of the whole-body overview image and/or of the data set of an examination region. An image acquisition with a 2D or 3D camera can also be implemented as a supplement to this or as an alternative to this.

In the image acquisition it should be noted that the resolution is sufficient to allow necessary first estimations to be made with regard to the anatomy or pathological findings or the selection of an examination region that can be relevant for the implementation of the following image acquisition. If applicable, a whole-body overview image can be generated on the basis of an anatomical atlas or using a computer program, for example using already-present data of the patient to be examined as an overview drawing.

With regard to the imaging medical examination apparatuses that can be used in the framework of the inventive method, no limitations exist as long as the acquisition of the data set of the examination region is possible with the necessary quality (if applicable) information with regard to the influence of the agent administered to the patient is to be acquired using the data. For example, a magnetic resonance exposure can thus be used in order to display a pathological finding while at the same time, using a contrast agent, biological distributions of the agent or a substance marked thereby can be monitored by magnetic resonance tomography. If applicable, a further imaging examination apparatus such as, for example, a PET apparatus can additionally be used, in particular for the determination of the influence of the agent. Temperature signals and the like can also be used in the framework of the imaging.

According to the invention, contrast agents and/or therapeutic agents and/or agents involved in such as cellular processes biological processes, in particular in metabolic processes and/or metabolic decomposition processes and/or gene activities, can be administered to the examination subject. For example, agents or elements that are used to achieve a warming effect also are suitable For example, heat is released given the formation of iron oxide caused by the administration of iron or iron compounds. Further such agents can release heat in interaction with other agents and substances. Since pathologically altered cells frequently exhibit different metabolic and gene activities than healthy cells, a conclusion (for example with regard to the assessment of a tumor) is possible by the acquisition of time-related information, for example in the framework of magnetic resonance acquisitions by an analysis of the acquired representation. Agents with radionuclides thus can be administered and shown, so conclusions of an existing disease are possible from an accumulation in specific regions or a decay later in the framework of an evaluation.

Variations of the contrast in an image exposure and/or a concentration of decomposition products and/or a signal-to-noise ratio of a measurement can be determined as information relating to the influence of the agent. These effects of the agent (such as a medicine and the like) can be determined, if applicable, before and after the application thereof, for example with a PET examination. For example, the decomposition of a contrast agent can be tracked by the change of its concentration and/or the concentration of the decomposition products, or the brightness of signals in the time period can be monitored.

The representation of the expanded whole-body overview image and/or the underlying acquired data set and/or the information relating to the influence of the agent and/or further data and/or information associated with the representation can be retained in a storage device for retrieval. For example, an already-created representation that is based on an image exposure after an administration of a contrast agent can be reused later by using, for a subsequent follow-up examination, the existing whole-body overview image as a whole-body overview image and the image data or new information with regard to the influence of an agent is incorporated into the already-supplemented whole-body overview image.

The acquired data set and associated data (such as, for example, a set of measurement parameters, information with regard to the acquired examination region as well as with regard to further characteristics) can likewise be retained for use in a further examination of the patient. In addition to this, the storage device can serve to access already-existing representations and other data in studies or acquisitions on other patients, if applicable in order to design the examination for the patients in a less elaborate manner. As information relating to the influence of the agent, the storage device can furthermore contain basic information such as the quantity, type or properties of the corresponding medicine or contrast agent, that can form the basic data for representation of the expanded whole-body overview image and can be helpful given a later continuation of a treatment or given the development of suitable medicines. Subsequent examinations, procedures or biopsies that occur in the same examination region can be implemented given the use of the same imaging medical examination apparatus using a first parameter set for the image exposure that exists in the storage device, and the resulting data set is consequently stored again.

In addition to the parameters relating to the image acquisition, further data and/or information associated with the representation that are stored can accordingly be data and/or information relating to the examination region and/or the examination subject and/or the quantity and/or type and/or properties and/or distribution of the administered agent and/or associated time changes. Further data or information can naturally be stored in the storage device as needed.

Later image acquisitions can be implemented dependent on the stored data and/or information and/or the stored parameters. For example, using the present data, the administered dose or the type of the administration of an agent can be changed in a later image acquisition (in the framework of a subsequent examination) when a very fast decay of the agent into its end products is apparent from the stored data. Stored parameters can be provided for another examination region (for example with small changes that are specific to this examination region) in order to nevertheless obtain comparable image exposures for mutual representation with regard to the whole-body overview image after an adaptation of the parameters.

A databank system with medical information (in particular regarding findings of one and/or more examination subjects and/or earlier applications of the and/or similar agents) can be accessed to determine and show the influence-related information. In addition to stored findings for these or other examination subjects, information about existing stem cells or information regarding the flow of a specific agent in the bloodstream of patients in general, or in a patient to be examined, can be stored in this databank system. The databank system, which can be a storage device for the expanded whole-body overview image, the data sets and further data or information associated with the representation as well as the behavior of an already-administered agent, can furthermore include data regarding the variation of a contrast or regarding pathological changes and the like, using which data information relating to the influence of the agent can be shown without the comprehensive measurements for acquisition of such an extensive representation actually having to be implemented in the present case.

Using the data of the databank system, a conclusion about the behavior of an agent in the body of a patient can be obtained without a simultaneous measurement of the examination subject in the manner that would be required for determination of this information by direct measurement being necessary. The stress for the patient thus can be reduced or, in some cases, the desired precision of the monitoring of the effect of an agent can be achieved more effectively.

At least one option relating to the representation of the supplemented whole-body overview image, in particular regarding the navigation between and/or correlation of different findings and further processing of the representation for a subsequent evaluation, can be provided on the image reproduction device and/or be established on the part of a user. If applicable, the possibility can be offered to vary the viewing angle for the whole-body overview image or to omit some expansions in the representation, for example when a large amount of displayed, influence-related information for the administered agent would unnecessarily complicate the representation.

The user can navigate as needed between different findings that form the basis of the whole-body overview image, and correlations can be determined for which (if applicable) an existing image processing program can be used. Furthermore, a possible error correction can be provided for selection. In addition, options can be provided that concern protocols or values to be acquired in the measurement (for example before and after the administration of the agent) as well as the identification of regions to be considered. For example, tools from the field of computer-aided design can be made available for post-processing of the expanded whole-body overview image, among other things for processing of surfaces. Furthermore, if applicable the individual data sets of the whole-body overview image can be accessed by image processing programs.

The options can be changed and/or created in the first place by a user as needed. Suitable user rights and input fields can be provided for this in a program.

Examples of such options are options for error correction given data and information and/or options for the display of the concentration of the agent and/or the display of further acquired data and information and/or options for identification of a pathological change and/or of surfaces and/or structures of the body of the examination subject.

According to the invention, a standardized report can be automatically created for representation of the expanded whole-body overview image, in particular dependent on data of a databank system. The expanded whole-body overview image or also further whole-body overview images can form a component of this standardized report with regard to earlier data acquisitions or acquisitions with other administered agents and the like. A structured overview of anatomical or pathological conditions, if applicable including further knowledge (for example from expert systems or rules systems (neural networks)) is thus possible with the inventive representation method. The report can be entirely written or can offer extension possibilities or input possibilities for a user while retaining the structuring.

At the image reproduction device, an option for selection of at least one further imaging medical examination apparatus and/or further examination type and/or of examination workflows for acquisition of further data set and/or for interaction with existing information systems can be provided to a user. Not only is the supplemented whole-body overview image shown to the user (such as a doctor or technician), but at the image reproduction device he or she can directly initiate the implementation of further steps for examination or data acquisition by, for example, changing the protocol used for the acquisition of a further data set. The representation of an option for interaction with existing information systems on the monitor or display of the user, moreover, allows access to information systems of comparable medical apparatuses or to specialists in the corresponding field. For example, a contact with an external expert can be established directly via the image reproduction device with the representation of the supplemented whole-body overview image, and if applicable the supplemented whole-body overview image can be sent to this external expert.

According to the invention, at least one examination apparatus and/or examination parameter can be suggested to a user for a further examination dependent on the data set and/or the influence-related information and/or the information from at least one of the aforementioned information systems. This suggestion can be likewise displayed on the image reproduction device in order to enable a selection to the user. For generation of the suggestion, a data processing device refers to, for example, an information system such as a databank system. An access is also possible to adaptive (trainable) systems and general knowledge-based systems from which information is determined that is, if applicable, linked with the present data set or the influence-related information. For example, the change of metabolic products can be tracked using the time relationship of the influence-related information and the change of pathological conditions can be tracked using different acquisitions of the examination region. From this the data processing system can develop a suggestion for subsequent examinations.

The data set and/or the influence-related information can be determined before and/or during and/or after the administration of the agent, in particular using the same parameters. The influences and effects of administered agents thus can be shown in the time curve in the whole-body overview image. The representation can ensue such that the present information is superimposed in the representation for the point in time before the administration of the agent, during the administration of the agent, or afterwards, if applicable using different representation types such as, for example, different colors; or various supplemented whole-body overview images with respective different supplements can be determined and shown with regard to a data set before and after the administration of the agent. Furthermore, a representation of the expanded whole-body overview image in a temporal workflow corresponding to the determination of the information or data sets is possible in order to thus obtain a sequence or a movie of the occurring changes. Identical or corresponding parameters for a measurement to be implemented or a simulation for the influence of the agent to be implemented can be used for a good comparison basis.

The representation on the image reproduction device can ensue using techniques for multi-dimensional representation. The use of multi-dimensional representation types or platforms enables the user to get a better spatial orientation and, if applicable, an improved and more comprehensive representation of the present data and information in order, for example, to use the representation for planning of subsequent examinations or for image-guided examinations.

Furthermore, the invention concerns a device for representation of at least one examination region of an examination subject as well as at least one item of information (which information is to be retained for a subsequent evaluation) relating to the intracorporeal influence of at least one agent to be administered to the examination subject, which device implements the method described above.

### DESCRIPTION OF THE DRAWINGS

FIG. 1 is a flowchart for an exemplary embodiment of the inventive method.

FIG. 2 illustrates the use of whole-body overview images in the inventive method.

FIG. 3 is a block diagram of a system in accordance with the invention.

### DESCRIPTION OF THE PREFERRED EMBODIMENTS

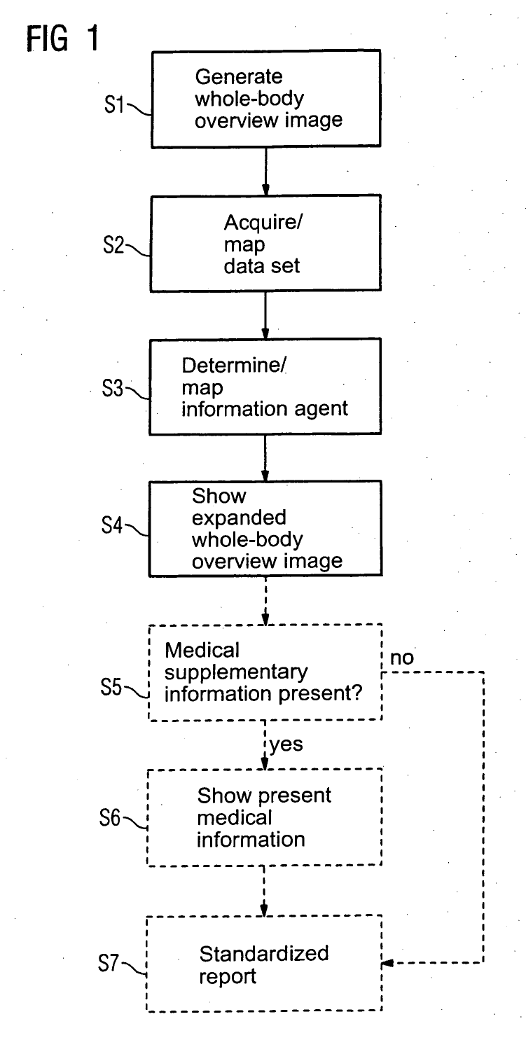

A flowchart of an embodiment of the inventive method is shown in FIG. 1. In accordance with the inventive method (which enables the simultaneous representation of multiple pathological findings with the effects of administered agents on a whole-body platform), a whole-body overview image of the examination subject is initially created in a step S1. This overview image can be created with an imaging medical examination apparatus or a camera or a computer program. Whole-body overview images that already exist from earlier examinations can be retrieved from a storage device for reuse in step S1.

In the following step S2, a data set associated with the examination region and acquired with at least one imaging medical examination apparatus and the acquired examination region are mapped to the whole-body overview image. For this purpose, it is possible to retrieve the whole-body overview image from a storage device for this purpose after the implementation of the acquisition has been initiated by a technician or, for example, a medical-technical assistant.

In the step S3, the influence of the intracorporeal agent that the patient has, for example, swallowed or that has been injected into the patient is determined in a time-related manner by access to corresponding information. The determination of the information already ensues before the acquisition of the agent and repeatedly after the administration of the agent in order to be able to track (update) the distribution or decomposition processes occurring in the body. The determination is implemented in the framework of the data acquisition with the imaging medical examination apparatus or another medical apparatus, for example by acquisition of magnetic resonance data. In a supplementary manner, the determination is implemented by access to corresponding data of a databank system using which a prediction with regard to the effect of the agent is possible in order to spare the patient possible stressful or unnecessary measurements. The information with regard to the effect of the medicine or of the contrast agent in the body of the patient is mapped to the whole-body overview image such that an expanded whole-body overview image results. Given a repeated determination of information with regard to the effect of the agent, this information can in turn be inserted into the whole-body overview image or into a further overview image.

In the following step S4, the whole-body overview image supplemented by the acquired data set and the determined information is finally shown on an image reproduction device. It is thus available for a subsequent evaluation, for example by a medical-technical assistant. The representation of the influence of the administered agent can thereby be designed such that the quantity, the type of the agent and its properties with its relation to the examined anatomical region are apparent from the representation.

In step S5, it is consequently checked whether medical information already exists, for example clinical realizations such as already-existing stem cells or previously-stored results with regard to pathological findings. If no such supplementary information exists in a storage device of a databank system, a standardized report regarding the expanded whole-body overview image is created from which multiple pathological findings can also arise if applicable by a repeated cycle of the method at this point.

If medical information exists, this is shown according to the step S6 in that, for example, the propagation of a contrast agent over time, a variation with regard to the contrast of a representation that is to be expected due to the present medical information or even variations in the pathological findings that result from previously-acquired image data of the examination region are likewise shown in the whole-body overview image. The distribution of contrast agents thus can also be inserted into the representation using already-present information. For example, it is thus possible to show a possible distribution of a further administered agent without a new measurement. The user can then navigate between different findings and correlate these and define the further processing via available options of a program means that are displayed on the image reproduction device. Finally, a standardized report is generated in turn.

With the inventive method, the speed and also the quality of a report generation regarding examinations with different examination apparatuses can thus be improved. Furthermore, a tool for development of image-supported technologies, in particular in the field of molecular imaging, is provided by the representation in the whole-body overview image.

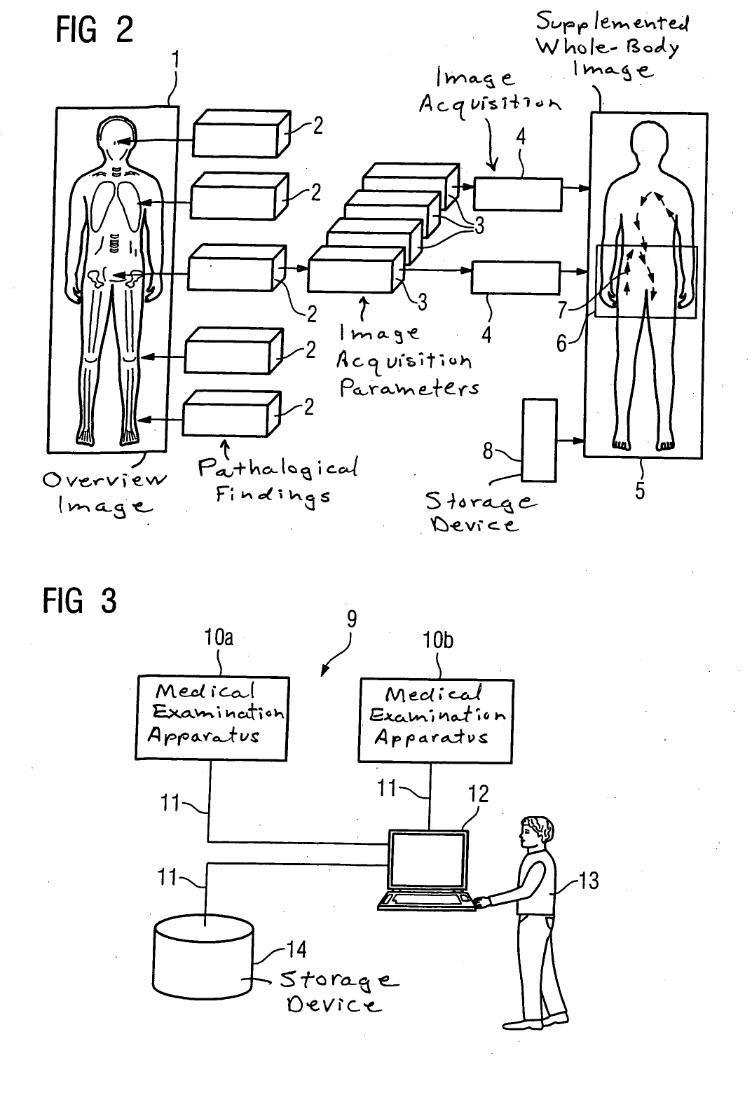

FIG. 2 shows a drawing regarding the use of whole-body overview images in an inventive method. For this, a whole-body overview image 1 is initially generated with an imaging medical examination apparatus. Different pathological findings 2 in different regions of the body which are shown in the whole-body overview image 1 arise from the whole-body overview image 1 in connection with data or corresponding suppositions already present beforehand.

With regard to the examination regions predetermined by the pathological findings 2, an acquisition of a data set is consequently generated with one of many available imaging medical examination apparatuses 3, here a magnetic resonance apparatus. The acquisition is connected with various available options and parameters that also concern the further processing of the acquired data sets. This connection of the image acquisition with parameters and the like should be expressed by the acquisition box 4. This concerns both the parameters that are used for the image acquisition with the examination apparatus 3, the type of the acquired data, whether (for example) relaxation data are acquired and processed, the target (goal) of the acquisition, thus the representation of a specific examination region and, if applicable, of a pathological finding 2 and the further processing of the image data. In the framework of the further processing, a further examination can be proposed and comparisons between different exposures can be conducted.

Information with regard to the intracorporeal influence of an agent administered to the patient is additionally determined with another examination apparatus 3. In the present case, this occurs via implementation of a positron emission measurement. An acquisition box 4 is likewise indicated for this measurement and the further processing of the respectively-acquired signal data. As a result, the acquired data set or, respectively, the time-related determined information regarding the administered agent are mapped to the whole-body overview image 1 such that a supplemented whole-body overview image 5 results that additionally shows the exposure 6 of the examination region and the information 7 pertaining to the effect of the agent. This information 7 is here shown as vector arrows that indicate the flow as well as the distribution of the administered agent. With these expansions, the whole-body overview image (which is based on standardized coordinates) can be used, for example, for planning a therapy. Furthermore, supplementary data such as medical information in the form of rules or knowledge can be drawn from a storage device 8 for expansion and representation of the whole-body overview image.

Finally, FIG. 3 shows an inventive device 9 that is fashioned for implementation of the method as illustrated in the preceding. The device 9 has imaging medical examination apparatuses 10a and 10b that are connected via data lines 11 with a computer 12 that includes an image reproduction device. The computer 12 serves for control of the imaging medical examination apparatuses 10a and 10b by a user 13 who operates these via the image reproduction device and a corresponding input unit.

Furthermore, a storage device 14 is provided that is likewise coupled to the computer 12 via a further data connection 11. The computer 12 has a system of different components such as various work calculators that are not shown in detail for clarity.

The imaging medical examination apparatus 10a is initially used to generate a whole-body overview image of a patient (not shown here), whereupon a data set of a specific examination region is produced with the same imaging medical examination apparatus 10a. The image acquisition is initiated by the user 13 via a program means of the computer 12. The whole-body overview image is shown to the user on his screen with the aid of the computer 12. The data regarding the examination region are subsequently mapped to the whole-body overview image by the computer 12 such that this whole-body overview image is supplemented.

The influence of an agent that has been administered to the patient is determined in a time-related manner in the framework of a conducted measurement with a further imaging medical examination apparatus 10b. This information transferred via the data connection 11 is likewise added into the whole-body overview image. Data of the storage device 14 with regard to earlier examinations as well as for a subsequent evaluation are additionally transferred (in part automatically, in part at the request of the user 13) to the computer 12 for expansion of the whole-body overview image and to supply the user 13 with further information. The supplemented whole-body overview image and the supplemental information retrieved from the storage device 14 are shown to the user 13 on his image reproduction device.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for representation of at least one examination region of an examination subject, together with at least one item of information relating to an intracorporeal influence of at least one agent administered to the examination subject, said item of information being retained for a subsequent evaluation, comprising the steps of:
generating a whole-body overview image of an examination subject;
with at least one medical imaging apparatus, acquiring a data set from an examination region of the examination subject and automatically electronically mapping, using said dataset, the examination region on the whole-body overview image;
determining at least one time-dependent item of information relating to an intracorporeal influence of an agent in the examination region, that has been administered to the examination subject, and automatically electronically mapping said item of information to the examination region in the whole-body overview image, thereby obtaining a supplemented whole-body overview image; and
visually representing the whole-body overview image at an image reproduction device.

2. A method as claimed in claim 1 comprising generating said whole-body overview image with said medical imaging apparatus.

3. A method as claimed in claim 1 comprising selecting said medical imaging apparatus from the group consisting of magnetic resonance, tomography apparatuses, x-ray apparatuses, computed tomography apparatuses, ultrasound apparatuses, and positron emission tomography apparatuses.

4. A method as claimed in claim 2 comprising selecting said medical imaging apparatus from the group consisting of magnetic resonance tomography apparatuses, x-ray apparatuses, computed tomography apparatuses, ultrasound apparatuses, and positron emission tomography apparatuses.

5. A method as claimed in claim 1 comprising generating said whole-body overview image from a source selected from the group consisting of a medical imaging apparatus, a camera, an anatomical atlas, and a computer program.

6. A method as claimed in claim 1 comprising administering said agent to the examination subject selected from the group consisting of contrast agents, therapeutic agents, agents involved in biological processes, agents involved in cellular biological processes, agents involved in metabolical processes, agents involved in metabolic decomposition processes, and agents involved in gene activities.

7. A method as claimed in claim 1 comprising determining, as said at least one item of information, information selected from the group consisting of contrast variations caused by the agent in an image exposure of the examination region, a concentration of decomposition products in the examination region caused by the agent, and a signal-noise ratio of a measurement in the examination region influenced by the agent.

8. A method as claimed in claim 1 comprising electronically storing, as stored information, at least one of the supplemented whole-body overview image, said dataset, and said at least one item of information, in a storage device for subsequent retrieval.

9. A method as claimed in claim 8 comprising retrieving at least some of said stored information from said storage device and subsequently acquiring a further dataset of the examination region using the retrieved stored information.

10. A method as claimed in claim 8 comprising determining further data selected from the group consisting of parameters relating to the acquisition of said dataset, medical data relating to the examination region, descriptive information relating to the examination region, medical data relating to the examination subject, descriptive information relating to the examination subject, a quantity of said agent, a type of said agent, properties of said agent, distribution of said agent in the examination region, and time-dependent changes of the influence of the agent in the examination region, and electronically storing said further data, as part of said stored information in said storage device, for subsequent retrieval.

11. A method as claimed in claim 10 comprising retrieving at least some of said stored information from said storage device and subsequently acquiring a further dataset of the examination region using the retrieved stored information

12. A method as claimed in claim 1 comprising determining said at least one item of information dependent upon information, accessed from a databank, selected from the group consisting of medical findings relating to other examination subjects, medical findings relating to earlier examinations of the examination subject, medical information relating to administration of agents identical to or similar to the agent administered to the examination subject.

13. A method as claimed in claim 1 comprising, at said image reproduction device, displaying at least one option relevant for a subsequent evaluation of the supplemented whole-body overview image.

14. A method as claimed in claim 13 comprising automatically electronically generating said option and automatically electronically displaying said option at said image reproduction device.

15. A method as claimed in claim 13 comprising generating said option by user interaction with said image reproduction device.

16. A method as claimed in claim 13 comprising selecting said option from the group consisting of error correction options, optional display of a concentration of the agent in the examination region, optional display of additional data concerning the examination region, optional display of further data concerning the examination subject, optional display of descriptive information concerning the examination subject, optional display of an identified pathological change in the examination subject, optional display of anatomical surfaces of the examination subject, and optional display of anatomical structures in the examination subject.

17. A method as claimed in claim 1 comprising automatically electronically generating a standardized report for representation of the supplemented whole-body overview image.

18. A method as claimed in claim 1 comprising, at said image reproduction device, making an option available for selection from the group consisting of selecting a further medical imaging apparatus, selecting a further examination type, and selecting a further examination protocol, for acquiring a further dataset from the examination subject.

19. A method as claimed in claim 1 comprising, at said image reproduction device, automatically electronically providing a suggestion, dependent on said at least one item of information, selected from the group consisting of a selection for an examination apparatus, and a suggestion for an examination parameter, for conducting a further examination of the examination subject.

20. A method as claimed in claim 1 comprising acquiring said dataset at a time selected from the group consisting of before administration of said agent, during administration of said agent, and after administration of said agent.

21. A method as claimed in claim 1 comprising acquiring each of said dataset and said at least one item of information substantially simultaneously at a time selected from the group consisting of during administration of said agent and after administration of said agent.

22. A method as claimed in claim 21 comprising acquiring said dataset and said at least one item of information using substantially identical acquisition parameters.

23. A method as claimed in claim 1 comprising representing said supplemented whole-body overview image at said image reproduction device in a multi-dimensional representation.

24. A system for representation of at least one examination region of an examination subject, together with at least one item of information relating to an intracorporeal influence of at least one agent administered to the examination subject, said item of information being retained for a subsequent evaluation, comprising:
a source of a whole-body overview image of an examination subject;
a medical imaging apparatus that acquires a data set from an examination region of the examination subject;
a computer, supplied with said data set and said whole-body overview image, that automatically maps, using said data set, the examination region on the whole-body overview image;
an acquisition device that acquires at least one time-dependent item of information relating to an intracorporeal influence of an agent in the examination region, that has been administered to the examination subject,
said computer automatically electronically mapping said item of information to the examination region in the whole-body overview image, thereby obtaining a supplemented whole-body overview image; and
an image reproduction device at which the whole-body overview image is visually represented.
